(19) United States
(12) Patent Application Publication
(10) Pub. No.: US 2025/0164776 A1
(43) Pub. Date: May 22, 2025

(54) IMAGE PICKUP UNIT, ENDOSCOPE, AND METHOD FOR MANUFACTURING IMAGE PICKUP UNIT

(71) Applicant: OLYMPUS MEDICAL SYSTEMS CORP., Tokyo (JP)

(72) Inventors: Masato MIKAMI, Tokyo (JP); Masateru SUZUKI, Tokyo (JP)

(73) Assignee: OLYMPUS MEDICAL SYSTEMS CORP., Tokyo (JP)

(21) Appl. No.: 18/945,007

(22) Filed: Nov. 12, 2024

Related U.S. Application Data
(60) Provisional application No. 63/599,712, filed on Nov. 16, 2023.

(51) Int. Cl. G02B 23/24; A61B 1/00; A61B 1/05; H04N 23/50; H04N 23/54

(52) U.S. Cl. CPC G02B 23/2484; H04N 23/54; H04N 23/555; A61B 1/0011; A61B 1/051

(57) ABSTRACT

An image pickup unit includes a circuit board including a first surface, a first side surface and a second side surface both intersecting the first surface, a depression including a first wall surface and a second wall surface, the depression dividing the first surface into a plurality of surfaces, a plurality of electronic components accommodated in the depression, and a first resin disposed in the depression, wherein the plurality of electronic components comprises a first electronic component disposed closest to the first side surface, a first distance between the first electronic component and the first wall surface, and the first distance is greater than a second distance between each of the plurality of electronic components other than the first electronic component and whichever wall surface is closer between the respective first wall surface and the second wall surface.

### RELATED APPLICATION DATA

This application is based on and claims priority under 35 U.S.C. § 119 to U.S. Provisional Application No. 63/599,712, filed on Nov. 16, 2023, the entire contents of which are incorporated herein by reference.

### BACKGROUND

### 1. Field

The present disclosure relates to an image pickup unit in which a plurality of electronic components are mounted between a planar circuit board and a variant circuit board, an endoscope including an image pickup unit in which a plurality of electronic components are mounted between a planar circuit board and a variant circuit board, and a method for manufacturing an image pickup unit in which a plurality of electronic components are mounted between a planar circuit board and a variant circuit board.

### 2. Description of the Related Art

Japanese Patent Application Laid-Open Publication No. 2017-23234 discloses an image pickup unit of an endoscope using a variant circuit board that is a three-dimensional circuit device. The image pickup unit includes a semiconductor package, a planar circuit board which is bonded to the semiconductor package and on which a plurality of electronic components are mounted, and a variant circuit board which is bonded to the planar circuit board and on which the plurality of electronic components are accommodated in a notch of the variant circuit board.

### SUMMARY

An image pickup unit according to an embodiment of the present disclosure includes: a circuit board including a first surface, a first side surface and a second side surface both intersecting the first surface, a depression including a first wall surface and a second wall surface, the depression dividing the first surface into a plurality of surfaces; a plurality of electronic components accommodated in the depression; and a first resin disposed in the depression, wherein the plurality of electronic components comprises a first electronic component disposed closest to the first side surface, a first distance between the first electronic component and the first wall surface, and the first distance is greater than a second distance between each of the plurality of electronic components other than the first electronic component and whichever wall surface is closer between the respective first wall surface and the second wall surface.

An endoscope according to an embodiment of the present disclosure includes an image pickup unit disposed in a distal end portion of an insertion portion, the image pickup unit including: a circuit board including a first surface, a first side surface and a second side surface both intersecting the first surface; a depression including a first wall surface and a second wall surface, the depression dividing the first surface into a plurality of surfaces; a plurality of electronic components accommodated in the depression; and a first resin disposed in the depression, wherein the plurality of electronic components comprises a first electronic component disposed closest to the first side surface, a first distance between the first electronic component and the first wall surface, and the first distance is greater than a second distance between each of the plurality of electronic components other than the first electronic component and whichever wall surface is closer between the respective first wall surface and the second wall surface.

A method for manufacturing an image pickup unit according to an embodiment of the present disclosure includes: fabricating a semiconductor package, a planar circuit board, and a circuit board including a first side surface and a second side surface, both the first side surface and the second side surface intersecting the planar circuit board, the circuit board including a depression including a first wall surface and a second wall surface; bonding a plurality of electronic components to the planar circuit board; a first distance between a first electronic component among the plurality of electronic components disposed closest to the first side surface and the first wall surface being greater than a second distance between each of the plurality of electronic components other than the first electronic component and whichever wall surface is closer between the respective first wall surface and the second wall surface; electrically connecting the semiconductor package to the planar circuit board; electrically connecting the planar circuit board to the circuit board; injecting resin between the first electronic component and the first wall surface; and curing the resin.

### BRIEF DESCRIPTION OF THE DRAWINGS

FIG. 1 is a perspective view of an image pickup unit according to a first embodiment;

FIG. 2 is an exploded view of the image pickup unit according to the first embodiment;

FIG. 3 is a rear view of a planar circuit board of the image pickup unit according to the first embodiment;

FIG. 4 is a side view of the image pickup unit according to the first embodiment;

FIG. 5 is a flow chart of a method for manufacturing the image pickup unit according to the first embodiment;

FIG. 6 is a perspective view for explaining the method for manufacturing the image pickup unit according to the first embodiment;

FIG. 7 is a sectional view for explaining the method for manufacturing the image pickup unit according to the first embodiment;

FIG. 8 is a sectional view of the image pickup unit according to the first embodiment;

FIG. 9 is a rear view of a planar circuit board of an image pickup unit according to a first modification of the first embodiment; and

FIG. 10 is a diagram showing a configuration example of an endoscope according to a second embodiment.

### DETAILED DESCRIPTION

### First Embodiment

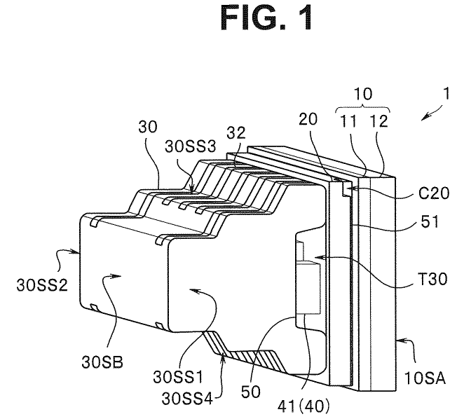

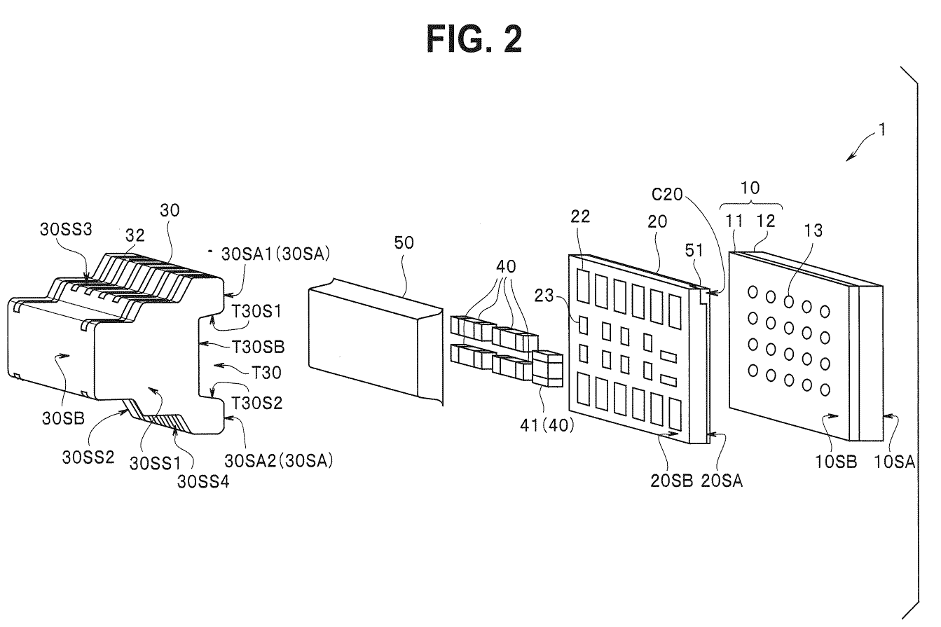

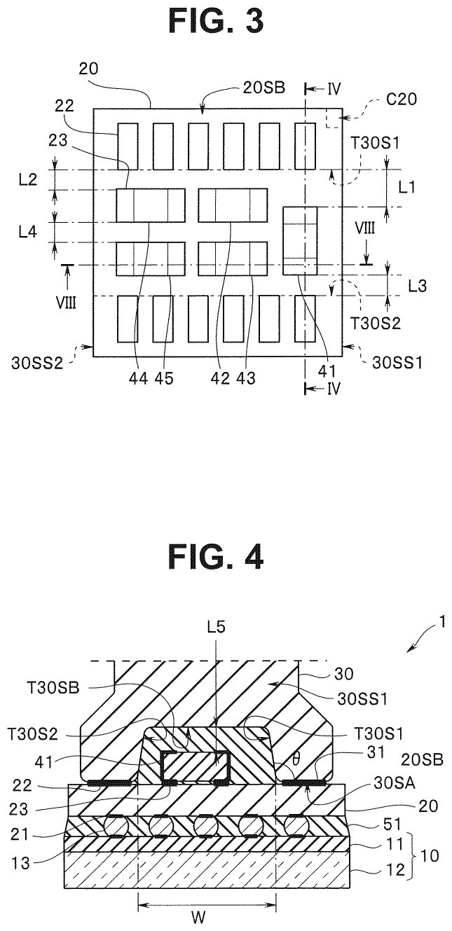

An image pickup unit 1 according to the present embodiment will be described with reference to FIGS. 1 to 4.

Note that the drawings based on the embodiments are schematic in nature. A relationship between a thickness and a width in each portion of the drawings, a ratio of thicknesses among respective portions, and the like differ from reality. Furthermore, even among the drawings, the drawings include portions having a relationship or a ratio among dimensions that differ from each other. Illustrations and assignments of reference signs with respect to a part of the components will be omitted. A direction in which light is incident will be referred to as a “front” and an opposite direction to the “front” will be referred to as a “rear”.

The image pickup unit 1 according to the present embodiment includes a semiconductor package 10, a planar circuit board 20, a variant circuit board 30, a plurality of electronic components 40, and resin 50.

The semiconductor package 10 includes a cover glass 12 and an image pickup device 11. The cover glass 12 includes a first surface 10SA. The image pickup device 11 with a front surface to which a rear surface of the cover glass 12 is glued includes a second surface 10SB being a rear surface. The image pickup device 11 containing silicon as a base material is an image sensor, such as a CMOS (complementary metal oxide semiconductor) light-receiving element or a CCD (charge coupled device).

A plurality of first electrodes 13 are arranged on the second surface 10SB. The first electrodes 13 are, for example, solder bumps. A drive signal and power are supplied to the image pickup device 11 via the plurality of first electrodes 13. In addition, a subject image received by the image pickup device 11 is converted into an electric signal and outputted as an image pickup signal via the plurality of first electrodes 13.

The semiconductor package 10 may in the image pickup device 11 include a stacked device in which a plurality of semiconductor devices that perform primary processing on the image pickup signal are stacked and the plurality of first electrodes 13 may be arranged on a rear surface of the stacked device. A lens unit in which a plurality of optical elements are stacked may be arranged on the first surface 10SA of the semiconductor package 10.

The planar circuit board 20 includes a third surface 20SA and a fourth surface 20SB on an opposite side to the third surface 20SA. A plurality of second electrodes 21 are arranged on the third surface 20SA. As shown in FIG. 4, each of the plurality of second electrodes 21 is bonded to a respective one of the plurality of first electrodes 13 of the semiconductor package 10. A plurality of third electrodes 22 and a plurality of fourth electrodes 23 are arranged on the fourth surface 20SB.

The plurality of electronic components 40 are surface-mounted to respective ones of the plurality of fourth electrodes 23. The plurality of electronic components 40 are, for example, chip capacitors that are approximate rectangular parallelopipeds. An approximate rectangular parallelopiped is a rectangular parallelopiped corners of which have been chamfered to form curved surfaces. In the image pickup unit 1, all of the plurality of electronic components 40 are size 0603 (long side: 0.6 mm, short side: 0.3 mm) chip capacitors. Each height of each of the plurality of electronic components is same.

The variant circuit board (circuit) 30 includes a fifth surface 30SA, a first side surface 30SS1 and a second side surface 30SS2, each of which being orthogonal to the fifth surface 30SA, and a sixth surface 30SB on an opposite side to the fifth surface 30SA. The variant circuit board 30 is a molded circuit device (MID: molded interconnect device) which is fabricated by injection molding and which includes a surface on which wirings and the like are arranged. A base material of the MID is epoxy, liquid crystal polymer, polyimide, polycarbonate, and the like. The variant circuit board 30 being an MID enables complex shapes to be readily manufactured.

The fifth surface 30SA of the variant circuit board 30 is divided into a first front surface 30SA1 and a second front surface 30SA2 by a depression T30 respectively including openings on the first side surface 30SS1 and the second side surface 30SS2. The depression T30 is a groove including a first wall surface T30S1, a second wall surface T30S2, and a bottom surface T30SB. In other words, the variant circuit board 30 includes a first arm portion including the first front surface 30SA1 and a second arm portion including the second front surface 30SA2, respective inner surfaces of the two arm portions being the first wall surface T30S1 and the second wall surface T30S2. A width W in an opening portion of the depression T30 or, in other words, a distance between the first front surface 30SA1 and the second front surface 30SA2 is 1.2 mm.

A plurality of fifth electrodes 31 are arranged on the fifth surface 30SA (the first front surface 30SA1 and the second front surface 30SA2). Each of the plurality of fifth electrodes 31 is bonded to a respective one of the plurality of third electrodes 22 of the planar circuit board 20.

The plurality of electronic components 40 arranged on the fourth surface 20SB of the planar circuit board 20 are accommodated in the depression T30 of the variant circuit board 30.

A plurality of wiring layers 32 extended from each of the plurality of fifth electrodes 31 are arranged on each of a third side surface 30SS3 and a fourth side surface 30SS4 which are orthogonal to the first side surface 30SS1 and the second side surface 30SS2 of the variant circuit board 30. A signal cable (not illustrated) is bonded to each of the ends of the plurality of wiring layers 32.

Resin 51 (second resin) is arranged in a gap between the semiconductor package 10 and the planar circuit board 20 or, in other words, in a bonded portion of the first electrodes 13 and the second electrodes 21. A notch C20 is located at a corner on the third surface 20SA in order to fill a narrow gap with the resin 51 in an efficient manner. The resin 51 in an uncured liquid state is injected into the gap from the notch C20 using a nozzle (not illustrated) and spreads between the semiconductor package 10 and the planar circuit board 20 due to interfacial tension.

The resin (first resin) 50 is arranged in the depression T30 of the variant circuit board 30 or, in other words, in a bonded portion of the fourth electrodes 23 and the plurality of electronic components 40.

The resin 50 is injected into the depression T30 as the resin 50 in an uncured liquid state in a same manner as the resin 51. However, it is not easy to fill spaces among the plurality of small electronic components 40 accommodated in the small depression T30 with the resin 50. For example, when disposing the plurality of electronic components 40 so as to be vertically and horizontally symmetrical, the plurality of electronic components 40 inhibit a flow of the resin 50. Therefore, there is a risk of creating voids where the resin 50 is not arranged and causing reliability to decline. There is also a risk of increasing a time period required to inject the resin 50. Furthermore, since openings that act as injection holes are small, there is a risk when injecting the resin 50 using the nozzle that the resin 50 may spill out to a periphery of the depression T30 and increase an outer diameter of the image pickup unit 1.

The image pickup unit 1 includes the following features in the disposition of the plurality of electronic components 40 accommodated in the depression T30 and the like.

A long side of a first electronic component 41 among the plurality of electronic components that is closest to the first side surface 30SS1 is disposed parallel to the first side surface 30SS1. A first distance L1 (0.4 mm) from the first electronic component 41 to the first wall surface T30S1 is greater than a third distance L3 (0.2 mm) from the first electronic component 41 to the second wall surface T30S2. In other words, the image pickup unit 1 secures an opening to be used as an injection hole when injecting the resin 50 on a side of the first side surface 30SS1.

Long sides of a plurality of electronic components 42 to 45 other than the first electronic component 41 are disposed perpendicular to the first side surface 30SS1.

A maximum distance among distances between the plurality of respective electronic components 42 to 45 other than the first electronic component 41 and whichever wall surface is closer between the respective first wall surface T30S1 and the second wall surface T30S2 is referred to as a second distance L2.

The electronic component 42 and the electronic component 44 are closer to the first wall surface T30S1 than to the second wall surface T30S2. The distance from the electronic component 42 and the electronic component 44 to the first wall surface T30S1 is the second distance L2 (0.2 mm). Note that the electronic component 43 and the electronic component 45 are closer to the second wall surface T30S2 than to the first wall surface T30S1. The distance from the electronic component 43 and the electronic component 45 to the second wall surface T30S2 is the same as the second distance L2.

In addition, a maximum distance among distances among the plurality of electronic components 40 (41 to 45) is referred to as a fourth distance LA. The distance between the electronic component 42 and the electronic component 43 is the fourth distance LA (0.25 mm). The distance between the electronic component 44 and the electronic component 45 is also the fourth distance LA. The distance between the electronic component 43 and the electronic component 45 and the distance between the electronic component 44 and the electronic component 42 are also 0.2 mm being shorter than the fourth distance LA.

Note that a fifth distance L5 being a distance between the plurality of electronic components 40 (41 to 45) and the bottom surface T30SB of the depression T30 is 0.25 mm.

In the image pickup unit 1 configured as described above, the first distance L1 (0.4 mm) between the first electronic component 41 and the first wall surface T30S1 is longer than widths of a plurality of other gaps (the second distance L2, the third distance L3, the fourth distance LA, and the fifth distance L5) in an internal space of the depression T30.

Furthermore, as shown in FIG. 4, the first wall surface T30S1 and the second wall surface T30S2 of the depression T30 of the variant circuit board 30 are inclined with respect to the fifth surface 30SA. Specifically, an angle θ of the first wall surface T30S1 and the second wall surface T30S2 relative to the fifth surface 30SA is more than 90 degrees and is 100 degrees. In other words, an interval between the first wall surface T30S1 and the second wall surface T30S2 (a width of the depression T30 being a notch) W decreases toward the bottom surface T30SB.

As will be described later, in the image pickup unit 1, the resin 50 injected between the first electronic component 41 and the first wall surface T30S1 efficiently spreads through the internal space of the depression T30 due to interfacial tension. Therefore, since voids in which the resin 50 is not arranged are less likely to be created in the depression T30, the image pickup unit 1 has high reliability.

### Method for Manufacturing Image Pickup Unit

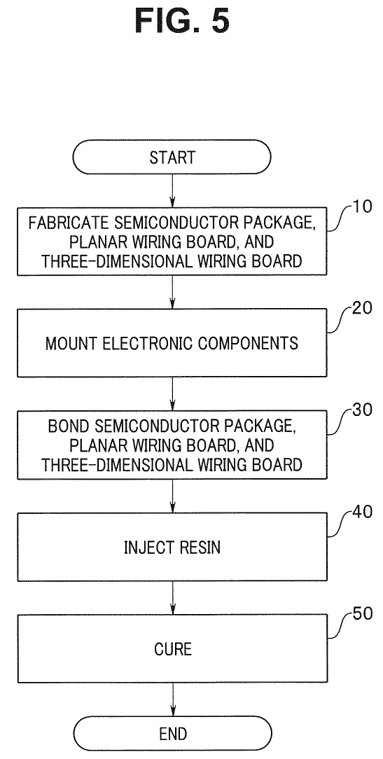

A method for manufacturing the image pickup unit 1 will be described along a flow chart shown in FIG. 5.

### Step S10

The semiconductor package 10, the planar circuit board (planar wiring board) 20, and the variant circuit board (three-dimensional wiring board) 30 are fabricated.

The semiconductor package 10 includes the first surface 10SA and the second surface 10SB on an opposite side to the first surface 10SA. The semiconductor package 10 includes the image pickup device 11 and the cover glass 12 and the plurality of first electrodes 13 are arranged on the second surface 10SB.

The planar circuit board 20 includes the third surface 20SA and the fourth surface 20SB on an opposite side to the third surface 20SA. The plurality of second electrodes 21 are arranged on the third surface 20SA, each of the plurality of second electrodes 21 being bonded to respective ones of the plurality of first electrodes 13. The plurality of third electrodes 22 and the plurality of fourth electrodes 23 are arranged on the fourth surface 20SB.

The variant circuit board 30 includes the fifth surface 30SA and the first side surface 30SS1 and the second side surface 30SS2 which are orthogonal to the fifth surface 30SA. The fifth surface 30SA is divided into the first front surface 30SA1 and the second front surface 30SA2 by the depression T30 respectively including openings on the first side surface 30SS1 and the second side surface 30SS2. The depression T30 being a notch includes the first wall surface T30S1, the second wall surface T30S2, and the bottom surface T30SB. The interval W between the first wall surface T30S1 and the second wall surface T30S2 decreases toward the bottom surface T30SB. The plurality of fifth electrodes 31 are arranged on the fifth surface 30SA.

Note that the angle θ of the first wall surface T30S1 and the second wall surface T30S2 relative to the fifth surface 30SA can be more than 90 degrees and less than 120 degrees and can be more than 95 degrees and less than 110 degrees. When the angle θ is within the ranges described above, the necessary number of the electronic components 40 can be accommodated in the depression T30 and, at the same time, the depression T30 can be filled with the resin 50 in an efficient manner.

### Step S20

The plurality of electronic components 40 are bonded to the plurality of fourth electrodes 23 of the planar circuit board 20.

The closest electronic component to the first side surface 30SS1 among the plurality of electronic components 40 is the first electronic component 41. As described earlier, the plurality of electronic components 40 are disposed in such a manner that the first distance L1 between the first electronic component 41 and the first wall surface T30S1 is longer than widths of a plurality of other gaps (the second distance L2, the third distance L3, the fourth distance L4, and the fifth distance L5) in the internal space of the depression T30.

### Step S30

Each of the plurality of first electrodes 13 of the semiconductor package 10 is bonded to a respective one of the plurality of second electrodes 21 of the planar circuit board 20. Each of the plurality of third electrodes 22 of the planar circuit board 20 is bonded to a respective one of the plurality of fifth electrodes 31 of the variant circuit board 30.

Note that the semiconductor package 10 and the planar circuit board 20 may be bonded to each other prior to step S20.

### Step S40

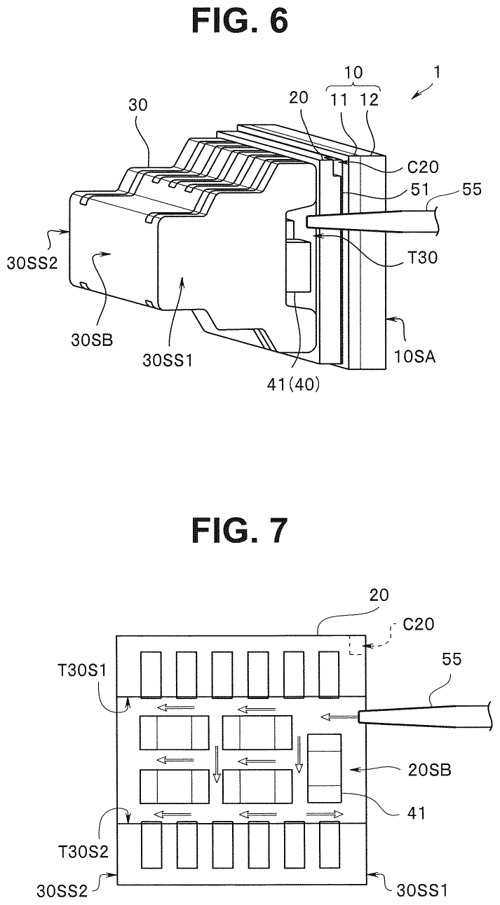

As shown in FIG. 6, the resin 50 is injected into the depression T30 using a nozzle 55 from between the first electronic component 41 and the first wall surface T30S1.

The first distance L1 (0.4 mm) between the first electronic component 41 and the first wall surface T30S1 is longer than widths of a plurality of other gaps (the second distance L2, the third distance L3, the fourth distance L4, and the fifth distance L5) in the internal space of the depression T30. Therefore, as shown in FIG. 7, the resin 50 spreads into a plurality of gaps in the internal space of the depression T30 due to interfacial tension.

Note that the first distance LI can be more than 0.3 mm to enable the resin 50 to be injected in an efficient manner. The second distance L2, the third distance L3, the fourth distance L4, and the fifth distance L5 can be less than 0.3 mm to enable the resin 50 to readily spread due to interfacial tension.

### Step S50

The resin 50 is subjected to a curing process. In the effect process, for example, a thermosetting method or a combination method of ultraviolet curing and thermosetting is used.

According to the present manufacturing method, not only can the resin 50 be reliably filled but the resin 50 can also be filled in a short amount of time. Furthermore, there is no more risk of the resin 50 spilling out to the periphery of the depression T30 and increasing the outer diameter of the image pickup unit 1 when the resin 50 is being injected.

Although a description has been omitted, in step S40, the resin 51 is injected into a gap between the semiconductor package 10 and the planar circuit board 20 and, in step S50, the resin 51 is subjected to a curing process.

For efficiency, the injection of the resin 50 and the injection of the resin 51 are consecutively performed. For this reason, the notch C20 of the planar circuit board 20 to be an injection port of the resin 51 can be located on a side of the first side surface 30SS1 where the injection port of the resin 50 is located.

Note that, the resin 50 and the resin 51 can be the same resin. In the image pickup unit 1 in which the resin 50 is arranged in the gap between the semiconductor package 10 and the planar circuit board 20, the resin 50 fills the notch C20 in a corner on the side of the first side surface 30SS1 of the third surface 20SA of the planar circuit board 20 having been injected with the resin 50.

In other words, the image pickup unit 1 can include a notch to be filled by the resin 50 in a corner on the side of the first side surface 30SS1 of the third surface 20SA of the planar circuit board 20.

### Modifications of First Embodiment

Image pickup units 1A and 1B according to modifications of the first embodiment are similar to the image pickup unit 1 and produce the same effects as the image pickup unit 1. Therefore, components with same functions as in the image pickup unit 1 will be assigned same reference signs as in the image pickup unit 1 and descriptions of such components will be omitted.

### First Modification of First Embodiment

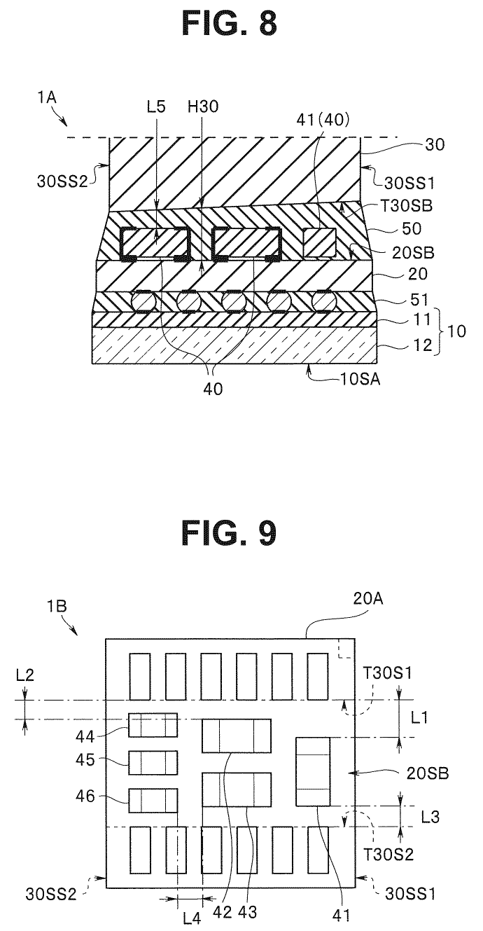

In the image pickup unit 1A according to the present modification shown in FIG. 8, the bottom surface T30SB of the depression T30 of the variant circuit board 30 is inclined with respect to the fifth surface 30SA. In other words, further toward the side of the second side surface 30SS2 from the side of the first side surface 30SS1, the fifth distance L5 (a depth H30 of the depression T30) being the distance between the plurality of electronic components 40 and the bottom surface T30SB comes continuously shorter.

Therefore, in the image pickup unit 1A, the resin 50 injected from the side of the first side surface 30SS1 more readily spreads to the side of the second side surface 30SS2 as compared to the image pickup unit 1.

### Second Modification of First Embodiment

In the image pickup unit 1B according to the present modification shown in FIG. 9, the plurality of electronic components 40 included in the image pickup unit 1B have different sizes and the first electronic component 41 is the largest among the plurality of electronic components 40.

The electronic components 41, 42, and 43 are size 0603 (long side: 0.6 mm, short side: 0.3 mm) chip capacitors. Electronic components 44 to 46 are size 0402 (long side: 0.4 mm, short side: 0.2 mm) chip capacitors.

A basic disposition of the plurality of electronic components 40 in the image pickup unit 1B is set to same conditions as the disposition of the plurality of electronic components 40 in the image pickup unit 1. However, the electronic component that is closest to the first side surface 30SS1 is the largest first electronic component 41 among the plurality of electronic components 40 (41 to 46).

In addition, the first distance L1 (0.4 mm) between the first electronic component 41 and the first wall surface T30S1 is longer than widths of the plurality of other gaps (the second distance L2, the third distance L3, the fourth distance L4, and the fifth distance L5) in the internal space of the depression T30.

Note that the image pickup unit according to the present disclosure may include the plurality of electronic components 40 with three or more different sizes. In addition, the plurality of electronic components 40 are not limited to chip capacitors but may include a plurality of electronic components with different functions such as chip inductors and IC chips.

### Second Embodiment

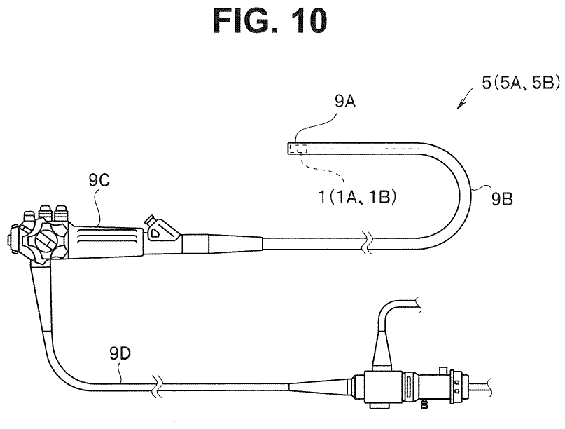

An endoscope 5 (5A, 5B) according to the present embodiment shown in FIG. 10 includes an insertion portion 9B with a distal end portion 9A in which the image pickup unit 1 (1A, 1B) is arranged, an operation portion 9C arranged on a proximal end side of the insertion portion 9B, and a universal cord 9D that extends from the operation portion 9C.

Since the endoscope 5 includes the image pickup unit 1 (1A, 1B) which has high reliability and which can be readily manufactured, the endoscope 5 also has high reliability and can be readily manufactured.

The present disclosure is not limited to the respective embodiments and modifications described above and various changes, alterations, and applications are possible without deviating from the scope of the gist of the disclosure.

What is claimed is:

1. An image pickup unit, comprising:
a circuit board including:
a first surface;
a first side surface and a second side surface both intersecting the first surface;
a depression including a first wall surface and a second wall surface, the depression dividing the first surface into a plurality of surfaces;
a plurality of electronic components accommodated in the depression, and
a first resin disposed in the depression, wherein
the plurality of electronic components comprises a first electronic component disposed closest to the first side surface,
a first distance between the first electronic component and the first wall surface, and
the first distance is greater than a second distance between each of the plurality of electronic components other than the first electronic component and whichever wall surface is closer between the respective first wall surface and the second wall surface.

2. The image pickup unit according to claim 1, wherein the first distance is greater than a third distance between the first electronic component and the second wall surface.

3. The image pickup unit according to claim 2, wherein the first distance is greater than a fourth distance being a maximum distance among a plurality of distances among the plurality of electronic components.

4. The image pickup unit according to claim 3, wherein the first distance is greater than a fifth distance being a maximum distance among a plurality of distances between the plurality of electronic components and a bottom surface of the depression.

5. The image pickup unit according to claim 4, wherein the bottom surface of the depression is inclined with respect to the first surface in such a state that the fifth distance decreases toward a side of the second side surface from a side of the first side surface.

6. The image pickup unit according to claim 1, wherein a long side of the first electronic component is disposed parallel to the first side surface and a long side of each of the plurality of electronic components other than the first electronic component is disposed perpendicular to the first side surface.

7. The image pickup unit according to claim 1, wherein the plurality of electronic components have a same size each other.

8. The image pickup unit according to claim 1, comprising
at least two electronic components of the plurality of electronic components have different sizes, wherein
the first electronic component is largest among the at least two electronic components.

9. The image pickup unit according to claim 1, wherein each height of each of the plurality of electronic components is same.

10. The image pickup unit according to claim 1, wherein a distance between the first wall surface and the second wall surface decreases toward the bottom surface.

11. The image pickup unit according to claim 1, further comprising:
a sensor configured to pick up an image; and
a planar circuit board including a second surface and a third surface opposite to the second surface, the planar circuit board including:
a first plurality of electrodes disposed on the second surface, the first plurality of electrodes being electrically connected to the sensor; and
a second plurality of electrodes disposed on the third surface, each of the plurality of electronic components being bonded to respective ones of the second plurality of electrodes.

12. The image pickup unit according to claim 11, wherein the planar circuit board further comprising:
a notch located at a corner on the second surface; and
a second resin disposed in the notch.

13. An endoscope comprising:
an image pickup unit disposed in a distal end portion of an insertion portion,
the image pickup unit including:
a circuit board including:
a first surface;
a first side surface and a second side surface both intersecting the first surface;
a depression including a first wall surface and a second wall surface, the depression dividing the first surface into a plurality of surfaces;
a plurality of electronic components accommodated in the depression; and
a first resin disposed in the depression, wherein
the plurality of electronic components comprises a first electronic component disposed closest to the first side surface,
a first distance between the first electronic component and the first wall surface, and
the first distance is greater than a second distance between each of the plurality of electronic components other than the first electronic component and whichever wall surface is closer between the respective first wall surface and the second wall surface.

14. The endoscope according to claim 13, wherein the first distance is greater than a third distance between the first electronic component and the second wall surface.

15. The endoscope according to claim 14, wherein the first distance is greater than a fourth distance being a maximum distance among a plurality of distances among the plurality of electronic components.

16. The endoscope according to claim 15, wherein the first distance is greater than a fifth distance being a maximum distance among a plurality of distances between the plurality of electronic components and a bottom surface of the depression.

17. The endoscope according to claim 16, wherein the bottom surface of the depression is inclined with respect the first surface in such a state that the fifth distance decreases toward a side of the second side surface from a side of the first side surface.

18. The endoscope according to claim 13, further comprising:
a sensor configured to pick up an image; and
a planar circuit board including a second surface and a third surface opposite to the second surface, the planar circuit board including:
a first plurality of electrodes disposed on the second surface, the first plurality of electrodes being electrically connected to the sensor; and
a second plurality of electrodes disposed on the third surface, each of the plurality of electronic components being bonded to respective ones of the second plurality of electrodes.

19. A method for manufacturing an image pickup unit comprises:
fabricating a semiconductor package including a sensor configured to pick up an image, a planar circuit board, and a circuit board including a first side surface and a second side surface, both the first side surface and the second side surface intersecting the planar circuit board, the circuit board including a depression including a first wall surface and a second wall surface;
bonding a plurality of electronic components to the planar circuit board,
a first distance between a first electronic component among the plurality of electronic components disposed closest to the first side surface and the first wall surface being greater than a second distance between each of the plurality of electronic components other than the first electronic component and whichever wall surface is closer between the respective first wall surface and the second wall surface;
electrically connecting the semiconductor package to the planar circuit board;
electrically connecting the planar circuit board to the circuit board;
injecting resin between the first electronic component and the first wall surface; and
curing the resin.

20. The image pickup unit according to claim 1, wherein the first resin fills the depression.
